(19) United States
(12) Patent Application Publication
(10) Pub. No.: US 2004/0192777 A1
(43) Pub. Date: Sep. 30, 2004

(54) Novel anticancer diterpene compounds, process and uses thereof

(75) Inventors: Mohammad Yaseen Dar, Srinagar (IN); Mohammad Yousuf, Srinagar (IN); Mushtaq A. Qurishi, Srinagar (IN); Anees A. Ansari, Srinagar (IN); Shamshad Ahmad, Srinagar (IN); Abdul Sami Shawl, Jammu (IN); Ajit Kumar Saxena, Jammu (IN); Gulam Nabi Qazi, Jammu (IN)

Correspondence Address: MERCHANT & GOULD PC, P.O. BOX 2903, MINNEAPOLIS, MN, 55402-0903, US

(73) Assignees: COUNCIL OF SCIENTIFIC AND INDUSTRIAL, New Delhi (IN); REGIONAL RESEARCH INSTITUTE OF UNANI MEDICINE (Central Council of Research in U.M. New Delhi), Srinagar (IN)

(21) Appl. No.: 10/401,404

(22) Filed: Mar. 28, 2003

(51) Int. Cl. C07C 61/29; A61K 31/22

(52) U.S. Cl. 514/559; 562/403

(57) ABSTRACT

Two compounds namely ent-pimmarane 8(14),15-diene-19 oic acid (compound 1) and ent-pimmarane 7(8),9(11),15-diene-19 oic acid (compound 2) were isolated from Lavatera cachmeriania, these compounds, in vitro, significantly inhibited the growth of number of human cancer cell lines (CNS : SK-N-MC, Colon : HT-29, Lung : A-549 , Liver: Hep-2, Ovary: OVCAR-5, Prostate: PC-3) representing different organs.

### FIELD OF THE INVENTION

The present invention relates to two compounds namely ent-pimmarane 8(14),15-diene-19-oic acid (compound 1) and ent-pimmarane 7(8),9(11),15-diene-19 oic acid (compound 2), its isolation from Lavatera cachmeriania. The compounds significantly in vitro inhibited the growth of number of human cancer cell lines such as CNS : SK-N-MC, Colon: HT-29, Lung : A-549, Liver: Hep-2, Ovary: OVCAR-5, and Prostate PC-3, representing different organs.

### BACKGROUND OF THE INVENTION

Cancer or neoplasm is the malignant new growth anywhere and elsewhere in the body system. It is characterized by unregulated proliferation of cells and a growing public health problem whose estimated worldwide new incidence is about six million cases per year. In most of the countries, cancer is second only to heart disease as cause of death. It can arise in any organ of the body but some sites are prone to attack than others such as breast, throat, intestine, leukocytes etc. Each cancer is propagated from a single cell that cut at some stage, it becomes free from its territorial restraints, which form a family of cells that multiply without limits and appear in the form of tumors.

During the transition from normal cell to a tumor cell a profound and heritable change occurs which allows a tumor cell to determine its own activities largely irrespective of the laws that govern so precisely the growth of all normal cells in an organism. This newly acquired property, which is known as autonomy, is the most important single characteristic of tumor cells since without it there would be no tumors. Another distinguishing characteristic of tumor cells is their lack of perfect form of function. The differences that exist between cancer and normal cells are that, compared to normal cells, cancer cells have a) low pH b) greater free radical character c) tumor produced hormone peptides d) tumor associated antigens e) lower calcium ion and higher potassium ion concentration f) different potassium isotope ratios g) elevated amounts of methylated nucleotides h) higher concentrations of plasma microproteins and mucopolysacharides i) greater need of exogenous zinc and j) high biowater content.

Many of the gross causes of cancer, such as dietary, environmental, occupational exposure to certain chemical substances or forms of electromagnetic radiation, have been elucidated through epidemiological studies. It is imperative, therefore, that they be identified and eliminated from the environment in so far as that is possible in, modern industrial societies.

In the annals of therapy, a quest to conquer, the impasse of cancer has been always fascinated, by and large, all disciplines of scientific community, especially natural product chemists. In 19^{th} and 20^{th} century, lot of research work has been carried out to find out the driving force behind this dreadful disease as well as large number of drugs have been introduced to counter its menace. The plants have always fascinated the scientists and as a result number of drugs for different diseases including cancer have been isolated from the plants. It is worthwhile at this juncture to look briefly at a few most powerful chemotherapeutic agents, which have been of paramount importance to the mankind and also to the researches who have been actively involved in the synthesis and isolation of anticancer drugs.

The well known anticancer drugs from plants are vinca alkaloids, vinblastine and vincristine from Catharanathus roseus (Smets, L. A; 1994, Anticancer Drugs, 5 : 3-9) and podophyllotoxin from podophyllum peltatum (Royal society of chemistry, London, 1997) which is not used but its two semi synthetic glycosides, Etoposide and Tenioposide are in clinical use (F. Gago, I drugs, 1999, 2, 309, K. Z. Rana et. al. Pharmacotherapy, 1999, 19, 35). The later addition in the list of anticancer drugs from plants is paclitaxel (Rowinsky, E. K., and Donehowar, R. C. Paclitaxel (Taxol). N. Engl. J. Med. 1995, 332: 1004-1014). The complex diterpene, Taxol (A. Endo, J. Med. Chem; 1985, 28, 401), was isolated from the bark of Taxus brevifolia. Paclitaxel along with several key precursors (baccatins), occurs in the leaves of various taxus species, such as Docetaxel (A. Endo. J. Antibiot, 1979, 32, 852), has provided a major, renewable natural source of this important class of drugs.

With the above background we focussed our attention towards the identification and isolation of potent cytotoxic agent(s) from plants. There is hardly any report regarding isolation and characterisation of compounds from Lavatera cachmeriania. (family malvaceae). Lavatera cachmeriania is a semi-evergreen, woody-based perenial, with wiry stems. The flowers are silky and clear pink in colour. The leaves are mid-green, ivy shaped, and downy. Lavatera cachmeriania is described as a minor variant of L. thuringiaca; however the foliage is distinctive. The 20-25 species of Lavatera have a broadly Mediterranean distribution stretching to south west Britain, the canary is Abyssinia central Asia and Kashmir with out lying species in Australia and eastern Siberia

However, this plant genus is known to have cathartic and anti-inflammatory activities (Nazionale dele Ricerche, Economic botany, Bari, V. 25 (1) P-107 Oct; 1998). The isolated compounds from the plant genus are dihydromalvalic acid from the seed oils (Vickey and J. R., JAOCS, J. Am oil Chem Soc. Australia 1981, 58, 731-32), cyclopropene and dihydrostercuklic acids (fatty acids) From the plant species (Vickey and J. R., JAOCS, J. Am oil Chem. Soc. Australia 1980, 57(2), 87-91), cyclopenoid from the seeds (Lotti, G. Izzo, R., 1^{st} Ind. Agrar University, Agrar 1993, 11(9), 303-8). All these compounds exhibit activities other than anticancer.

### OBJECTS OF THE INVENTION

The main object of the present invention is to provide novel compounds from plant Lavatera cachmeriania useful for inhibiting growth of human cancer cell lines.

Another object of the invention is to characterize the novel compounds.

Another object of the invention is to evaluate the efficacy of the compounds against the human cancer cells.

Still another object of the invention is to provide a process for the isolation of the said compounds from Lavatera cachmeriania.

Yet another object of the invention is to provide a pharmaceutical composition for inhibiting growth of human cancer cell lines.

### SUMMARY OF THE INVENTION

Accordingly, the present invention provides two novel compounds namely ent-pimmarane 8(14),15-diene-19-oic acid identified as compound 1 and ent-pimmarane 7(8),9(11),15-diene-19 oic acid identified as compound 2. The present invention also provides a process for the isolation of the compound from plant Lavatera Cachmiriana. The invention also provides pharmaceutical compositions comprising effective amount of the compound 1 or compound 2 or combination thereof.

### DETAILED DESCRIPTION OF THE INVENTION

Accordingly, the present invention provides two new compounds i.e. compound 1 and compound (2) named as ent-pimmarane 8(14),15-diene-19 oic acid and ent-pimmarane 7(8),9(11),15 triene-19 oic acid respectively, isolated from plant Lavatera cachmeriania, having structural formula as shown below:

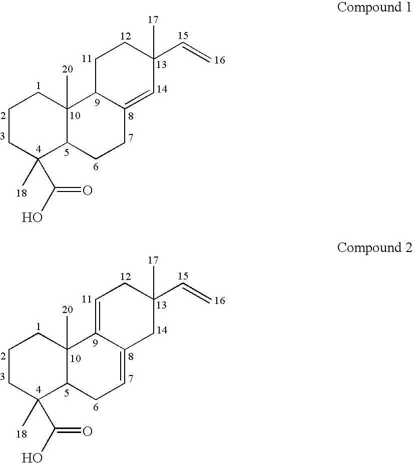

In an embodiment of the invention, the compound (1) is having the following characteristics:

### Compound (1)

m/z: 302(M+) IR₍νₘₐₓ₎: 3552-3228 (0-H); 1684 (C=0); 1598; 1410 and 910 (trisubstituted double bond) and 1264, 1024, 860 (di-substituted double bond as well) cm⁻¹ UV (λₘₐₓMeoH): 215 nm ¹H-NMR and ¹³C-NMR of compound (1):

TABLE 1
¹H-NMR (chemical shifts) of Compound 1 (200 MHz, CD Cl₃)

| | Integrated | | | |
|---|---|---|---|---|
| | No. of | | | |
| δ | Protons | Multiplicity | J in Hz | Assignment |
| 0.65 | 3 | S | — | H-18 |
| 1.01 | 3 | S | — | H-20 |
| 1.25 | 3 | S | — | H-17 |
| 2.16 | 2H | dd, br | 6, 5 | H-7 |
| 2.31 | 1H | dd, br | 6, 7 | H-9 |
| 4.90 | 1H | Dd | 6, 7 | Ha-16 |
| 4.95 | 1H | S | — | H-14 |
| 5.72 | 1H | Dd | 6, 7 | H-15 |

TABLE 2
¹³C-NMR of Compound 1, 500 MHz

| Carbon No. | δc | Carbon No. | δc |
|---|---|---|---|
| 1 | 39.3 | 11 | 19.6 |
| 2 | 19.3 | 12 | 36.5 |
| 3 | 37.98 | 13 | 38.5 |
| 4 | 44.1 | 14 | 128.0 |
| 5 | 56.2 | 15 | 147.2 |
| 6 | 24.2 | 16 | 113.0 |
| 7. | 35.8 | 17 | 29.4 |
| 8. | 138.0 | 18 | 29.2 |
| 9. | 50.6 | 19 | 184.5 |
| 10 | 38.56 | 20 | 13.3 |

In another embodiment of the invention, the Compound (2) is having the following characteristics.

m/z: 300(M⁺) IR₍νₘₐₓ₎ 3520-2880 (OH); 1678(CO); νₘₐₓ 1440; 1408; 1330; 1184; 1092; 910 and 756 (tri-substituted double bond) cm⁻¹ UV (λₘₐₓ^{MeoH}): 240 nm ¹H-NMR and ¹³C-NMR of compound (2):

TABLE 3
¹H-NMR chemical shifts of Compound 2 (200 MHz, CD Cl₃)

| | Integrated | | | |
|---|---|---|---|---|
| δ | No. of Protons | Multiplicity | J in Hz | Assignment |
| 0.65 | 3 | s | — | H-18 |
| 1.09 | 3 | s | — | H-20 |
| 110 | 3 | s | — | H-17 |
| 1.48-1.52 | 6H | m, br | — | H-1 |
| 1.38 | 1H | dd | 3.3, 7.3 | H-5 |
| 2.31 | 1H | dd, br | 5, 8 | Ha-6 |
| 5.62 | 1H | dd | 7, 4 | H-7 |
| 5.75 | 1H | dd | 7.3, 3.5 | H-11 |
| 2.14 | 1H | br, d | 5 | Ha-12 |
| 2.15 | 1H | d, br | 5 | Hb-12 |
| 2.04 | 2H | s, br | — | Ha-14 |
| 4.94 | 1H | t | 5, 2 | Hx-15 |
| 4.88 | 1H | d | 2.06 | Ha-16 |
| 4.91 | 1H | d | 5 | Hb-16 |

TABLE 4
¹³C-NMR of Compound 2, 500 MHz

| Carbon No. | δc | Carbon No. | δc |
|---|---|---|---|
| 1 | 39.6 | 11 | 138.4 |
| 2. | 19.6 | 12. | 38.9 |
| 3. | 36.2 | 13 | 38.3 |
| 4. | 44.4 | 14 | 30.1 |
| 5. | 56.5 | 15. | 147.5 |
| 6. | 32.3 | 16. | 113.3 |
| 7. | 120.3 | 17. | 29.5 |
| 8. | 146.18 | 18. | 29.7 |
| 9. | 138.2 | 19 | 185.2 |
| 10. | 39.6 | 20. | 14.1 |

One more embodiment of the invention provides a process for the isolation of compounds (1) and (2), namely ent-pimmarane 8(14),15-diene-19-oic acid (compound 1) and ent-pimmarane 7(8),9(11),15-diene-19 oic acid (compound 2), the said process comprising steps of:

(a) powdering the plant part of Lavatera Cachmiriana,

(b) defatting the powdered plant of step(a), with an organic solvent,

(c) extracting the defatted plant-material of step(b) with an alcoholic solvent to obtain alcoholic extract,

(d) evaporating alcoholic extract of step (c), under reduced pressure to obtain a dry extract,

(e) purifying the dried extract of step (d) silica gel column chromatography, eluting with a mixture of organic solvents, and

(f) pooling identical eluted fractions of step (e) on the basis of TLC analysis, concentrating the pooled fraction to reduced volume and crystallizing by adding an organic solvent to obtain the required compounds (1) and (2).

In an embodiment, the invention provides a process, wherein the plant part used for isolating the compounds is whole plant.

Another embodiment, the organic solvent used in step (b), is selected from petroleum ether or hexane, preferably petroleum ether.

Still another embodiment, the w/v ratio of plant material and organic solvent used in step (b) ranges from 1:20 to 25.

In another embodiment, the alcoholic solvent used in step (c) is C₁ to C₆ aliphatic alcohol selected from a group consisting of methanol, ethanol, isopropanol, butanol, pentanol and hexanol, preferably methanol. The w/v ratio of plant material and alcoholic solvent ranges from 1:15 to 20.

In yet another embodiment, the mixture of organic solvent used in step (e) for elution is a mixture of petroleum ether: ethylacetate, which ranges from 98:2 to 90:10 Yet another embodiment, petroleum ether:ethylacetate in the ratio 95:15 eluate compound (1) and petroleum ether:ethylacetate in the ratio 90:10 eluate provides compound (2).

Yet another embodiment, the organic solvent used in step (f) for crystallization, is petroleum ether.

One more embodiment of the invention provides use of compounds (1) and (2), for treating a said subject afflicted with cancer mainly of lungs, ovary, central nervous system (CNS), colon, prostrate, and cervix; said method comprising administering a pharmaceutically effective dosage of compounds (1) and/or (2) or a formulation containing compound (1) and/or (2) or a combination thereof to the said subject.

One more embodiment of the invention provides a pharmaceutical composition comprising an effective amount compound (1) named as ent-pimmarane 8(14),15-diene-19 oic acid or compound (2) named as ent-pimmarane 7(8),9(11),15 triene-19 oic acid or combination thereof, for treating a said subject afflicted with cancer selected from group consisting of lungs, ovary, central nervous system (CNS), colon, prostrate and cervix.

Another embodiment of the invention, the compounds (1) and/or (2) may be administered singly or in combination with pharmaceutically acceptable excipient.

Still another embodiment, the pharmaceutically acceptable excipient used may be an additive, carrier, diluent, solvent, filler, lubricant, excipient, binder or stabilizer.

Yet another embodiment, the compounds (1) and/or (2) or its formulation may be administered orally or systemically.

In yet another embodiment, the said compounds or its formulation administered to the pathological conditions arising from cancer afflicted lungs, ovary, central nervous system (CNS), colon, prostrate, and cervix.

In yet another embodiment, the said formulation is administered to subjects, which are selected from animals, mammals or humans; preferably humans.

One more embodiment, compound (1) or its formulation inhibits the growth of human lung cancer cells (A-549) in the range of 13 to 94% at a concentration range of 10 to 100 μg/ml.

Another embodiment, the compound (1) or its formulation inhibits the growth of human liver cancer cells (Hep-2) in the range of 18 to 91% at a concentration range of 10 to 100 ug/ml.

Yet another embodiment, the compound (1) or its formulation inhibits the growth of human ovary cancer cells (OVCAR-5) in the range of 0 to 76% at a concentration range of 10 to 100 μg/ml.

Yet another embodiment, the compound (1) or its formulation inhibits the growth of human CNS cancer cells (SK-N-MC) in the range of 0 to 88% at a concentration range of 10 to 100 g/ml.

Yet another embodiment, the compound (1) or its formulation inhibits the growth of human colon cancer cells (HT-29) in the range of 2 to 94% at a concentration range of 10 to 100 μg/ml.

Yet another embodiment, the compound (1) or its formulation inhibits the growth of human prostrate cancer cells (PC-3) in the range of 5 to 97% at a concentration range of 10 to 100 μg/ml.

Yet another embodiment, the compound (1) or its formulation inhibits the growth of human cervix cancer cells (Si Ha) in the range of 0 to 41% at a concentration range of 10 to 100 μg/ml.

One more embodiment of the invention relates to activity of the compound (2) or its formulation, which inhibits the growth of human lung cancer cells (A-549) in the range of 9 to 97% at a concentration range of 10 to 100 μg/ml.

Yet another embodiment, the compound (2) or its formulation inhibits the growth of human liver cancer cells (hep-2) in the range of 18 to 91% at a concentration range of 10 to 100 μg/ml.

Yet another embodiment, the compound (2) or its formulation inhibits the growth of human ovary cancer cells (OVCAR-5) in the range of 12 to 96% at a concentration range of 10 to 100 μg/ml.

Yet another embodiment, the compound (2) or its formulation inhibits the growth of human CNS cancer cells (SK-N-MC) in the range of 22 to 92% at a concentration range of 10 to 100 μg/ml.

Yet another embodiment, the compound (2) or its formulation inhibits the growth of human colon cancer cells (HT-29) in the range of 0 to 95% at a concentration range of 10 to 100 μg/ml.

One more embodiment of the invention relates to a pharmaceutical composition comprising effective amount of compound 1 or compound 2 or combination thereof for treating

The following examples are given by the way of illustration of the present invention and therefore should not be construed to limit the scope of the invention.

### EXAMPLE 1

### Isolation of Compound 1 & 2

1250 gms of Lavatera cachmiriana (whole plant material) were powdered and defatted with 15 litre of Pet. ether (60-80°). Defatted plant material was subjected to extraction with 14 litre of methanol in Soxhlet apparatus for about 48 hours. Methanol extract, so obtained, was vacuum dried (140 g). 140 g. Methanol extract was fractionated by column chromatography using Silica gel (mesh size 60-120). Column elution was carried out separately using 5% ethyl acetate in pet ether (A) leading to the isolation of compound 1, and 10% ethyl acetate in pet ether (B). Fraction B leads to the isolation of compound 2.

### EXAMPLE-1

(A). Fractionation with 5% Ethyl Acetate in Pet Ether(Isolation of Compound 1 and 2):

Fractions upto 1000 ml collected with 5% EtOAc in pet ether was further subjected to re-column chromatography. Elution of column was carried out with increasing polarity of ethyl acetate in pet ether with 2% and 5%. Fractions upto 1000 ml of 2% ethyl acetate showed presence of fatty acids and were discarded. Next fractions upto 12,000 ml of 5% ethyl acetate revealed the presence of an interesting spot on TLC, and volume of the fraction was reduced to 15 ml by distillation. Crystallization of the compound was done by using 25 ml pet ether and 5 ml ethyl acetate, pure compound was obtained and labeled as Compound 1 melting point 135-36° C.

(B). Fractionation with 10% Ethyl Acetate in Pet Ether:

Fraction of volume 1250 ml from bomb column were pooled and volume was reduced to 30 ml by distillation. This fraction revealed the presence of intricate mixture and further tried to separate them by re-column chromatography. Column elution was carried out with increasing polarity of ethyl acetate with 2% and 5% in pet ether. Fraction upto volume of 585 ml of 2% ethyl acetate did not reveal the presence of any spot and was discarded. Next fraction upto volume of 450 ml of 5% ethyl acetate shows single on TLC and volume was reduced to 15 ml by distillation. The compound was crystallized by using 5 ml of ethyl acetate and 25 ml of pet ether as the solvent, pure compound was obtained labeled as Compound 2 melting point 125-26° C.

### EXAMPLE 2

2 Kg of Lavatera cachmiriana plant material were powdered and Defatted with 22,000 ml of pet ether (60-80°). Defatted plant material was subjected to methanol extraction with 20, 000 ml of methanol in soxhlet apparatus type for about 48 hours. Methanol extract so obtained was vacuum dried and found to be weighing 210 gms. Methanol extract 210 gm was fractionated by column chromatography. Column elution was carried out separately using 5% ethyl acetate in pet ether (A) and 5% ethyl acetate in pet ether as the solvent.

(A) Fractionation with 5% ethyl acetate in pet ether:—Fractions up to 16000 ml collected with 5% EtO Ac in pet ether shows intricate mixture and needs to be separate them by re-column chromatography elution of column was carried out with increasing polarity of ethyl acetate in pet ether with 2% and 5%. Fractions up to 1200 ml of 2% ethyl acetate shows presence of fatty acids and were discarded. Next fraction up to 1500 ml of 5% shows single spot on TLC and volume of fraction was reduced to 10 ml by distillation. This fraction after crystallization with 30 ml of pet ether yielded a compound labeled as compound-1 melting point 135-36° C.

(B) Fractionation with 10% ethyl acetate in pet ether:—Fractions of volume 1400 ml from bomb column were pooled and volume was reduced to 210 ml by distillation. This fraction shows intricate mixture and needs to separate them by column chromatography. Column elution was carried out with 2% and 5%. Fraction up to volume of 800 ml of 2% ethyl acetate do not shows any spot and were discarded. Next fraction up to volume of 700 ml of 5% ethyl acetate shows single spot on TLC and volume was reduced to 10 ml by distillation. The compound was crystallized by using pet ether as solvent and yielded a compound labeled as compound-2 melting point 125-26° C.

### EXAMPLE 3

1500 gms of Lavatera cachmiriana plant material were powered and defatted with 20,000 ml of pet ether (60-80°). Defatted plant material was subjected to methanol extraction with 17,000 ml of methanol in soxhlet apparatus for about 48 hours. Methanol extract so obtained was vacuum dried and found to be weighing 175 gms. Methanol extract 175 gms was fractionated by column chromatography column elution was carried out separately using 3% ethyl acetate in pet ether (A) and 6% ethyl acetate in pet ether

(A) Fractionation with 5% ethyl acetate:—Fractions up to volume of 1350 ml collected with 2% EtO Ac fractions up to shows intricate mixture and needs to be separate them by re-column chromatography. Column elution was carried out by using 2% and 5% ethyl acetate in pet ether. Fractions up to volume of 900 ml of 2% ethyl acetate do not show any spot on TLC and were discarded. Fraction up to volume of 1200 ml shows presence of single spot on TLC and volume of fraction was reduced to 10 ml by distillation. This fraction after crystallization with 30 ml of pet ether yielding a compound labeled as compound-1 melting point 135-36° C.

(B) Fractionation with 10% ethyl acetate:—Fractions up to volume of 1050 ml shows presence of mixture and needs to separate them by re-column chromatography. The volume of fraction was reduced to 15 ml by distillation. Column elution was carried out by using 2% and 5% ethyl acetate. Fraction up to volume of 900 ml of 2% ethyl acetate do not show any spot and were discarded. Next fraction up to volume of 650 ml of 5% ethyl acetate shows single spot on TLC and volume was reduced to 10 ml by distillation. The compound was crystallized by using pet ether as solvent and yielding a compound labeled as compound-2 melting point 125-26° C.

The structure of Compound 1 was elucidated as ent-pimmarane-8(14),15-diene-19 oic acid and structure is annexed herewith. Compound 1 was observed as white crystalline solid. The LC-MS of compound-1 showed the molecular ion peak M⁺+Na at m/z 325.1, corresponding to the molecular mass at m/z 302 corresponding to C₂₀H₃₀O₂. The functional nature of two oxygen atoms in the form of carboxylic group was further revealed by the IR spectrum υₘₐₓ 3552-3228 cm⁻¹, (O—H) 1684 cm⁻¹ (C═O) 1598, 1410 and 910 cm⁻¹ (tri-substituted double bond) 1264, 1024, 860 cm⁻¹ (di-substituted double bond as well).

The ¹H-NMR spectrum (Table 1) of Compound 1 revealed the presence of an exocylic disubstituted double bond by displaying an ABX pattern of coupled protons at δ 4.90 (1H, dd, J=6, 7 Hz, Ha-16), 4.95 (1H, dd, J=6, 7 Hz, Hb-16) and 5.72 (1H, dd, J=6, 7 Hz, Hx-15). The spectrum contained a broad singlet at δ 5.14 (1H, H-15) confirming the presence of a tri-substituted double bond in the molecule.

Since the Compound 1 shows end absorption in UV spectrum, λₘₐₓ^{MeoH} 215 nm, it rules out the presence of conjugation in the molecule.

The presence of carboxyl function was confirmed by preparation of methyl ester of Compound 1 with diazomethane, whose ¹H-NMR spectrum contained the resonance signal due to carbonyl methoxy protons at δ63.63 (3H, s) and showed a downfield shift in the resonance signals of two methyls to δ0.99 (3H, s) and 1.19 (3H,s). Hence the structure assigned to the molecule of Compound 1 is annexed herewith.

Pharmaceutical Compositions

In accordance with the practice of the invention, pharmaceutical compositions employing the novel compounds of the invention with pharmaceutically acceptable carriers may be prepared. The pharmaceutical preparations of the invention are synergistic in nature and exhibit, surprising properties of the invention are synergistic in nature and exhibit surprising properties and effects. The proportion of the active ingredient to the carrier or additives may be in the range of 1:1 to 1:100 preferably, in the range of 1:1 to 1:10.

The compositions of this invention may contain physiologically acceptable diluents, fillers, lubricants, excipients, solvents, binders, stabilizers, and the like, diluents that may be used in the composition include but or are not limited to dicalcium phosphate, calcium sulphate, lactose, cellulose, kaolin, mannitol, sodium chloride, dry starch, powdered sugar and for prolonged release tablet-hydroxy propyl methyl cellsulose (HPMC). The binders that may be used in the composition include but or are not limited to starch, gelatin and fillers such as sucrose, glucose, dextrose and lactose.

Natural and synthetic gums used in the present invention are selected from sodium alginate, ghatti gum, carboxymethylcellsulose methylcellsulose, polyvinyl pyrrolidone and veegum. The excipient used in the present process is selected from microcrystalline cellsulose, calcium sulfate, dicalcium phosphate, starch, magnesium stearate, lactose, sucrose. Stabilizers used are polysaccharides such as acaciaa, agar, alginic acid, guar gum and tragacanth, amphotsics such as gelatin and synthetic and semi-synthetic polymers such as carbomer resins, cellsulose ethers and carboxymethyl chitin.

Solvents that may be used include but are not limited to Ringers solution, water, distilled water, dimethyl sulfoxide to 50% in water, propylene glycol (near or in water), phosphate buffered saline, balanced salt solution, glycol and other conventional fluids.

The compositions prepared in accordance with the practice of the invention may be administered to subjects in need thereof systematically.

Systemic administration refers to oral, rectal, nasal, transdermal and parental (i.e., intra muscular, intraperitoneal, subcutaneous or intravenous). In accordance with good clinical practice, it is preferred to administer the composition in a dose that will inhibit/prevent growth of cancerous cells without causing undue harmful side effects. The composition may be administered either alone or as a mixture with other therapeutic agents.

In Vitro Cytotoxicity of Compound 1 against Human Cancer Cell Lines:

The human cancer cell lines were obtained either from National center for cell science, Pune, India or National Cancer Institute, Frederick, MD, U.S.A. Cells were grown in tissue culture flasks in complete growth medium (RPMI-1640 medium with 2 mM glutamine, 100 μg/ml streptomycin, pH 7.4, sterilized by filtration and supplemented with 10% sterilized fetal calf serum and 100 units/ml penicillin before use) at 37° C. in an atmosphere of 5% CO₂ and 90% relative humidity in a carbon dioxide incubator. The cells at subconfluent stage were harvested from the flask by treatment with trypsin (0.05% trypsin in PBS containing 0.02% EDTA) and suspended in complete growth medium. Cells with cell viability of more than 97% by trypan blue exclusion technique were used for determination of cytotoxicity.

Compound 1 was dissolved in DMSO (dimethyl sulphoxide) to obtain a stock solution of 20 mg/ml. The stock solution was serially diluted with complete growth medium containing 50 μg/ml of gentamycin to obtain three working test solutions of 200, 60 and 20 μg/ml.

The suspension of human cancer cell lines of required cell density in complete growth medium was prepared and cell suspension of each cell line was placed in 96-well tissue culture plate (100 μl per well) for control, Compound 1 and positive control(s). The blank wells for each cell line and concentration of test material were also included that contained equivalent amount of complete growth medium only. The plates were incubated at 37° C. in an atmosphere of 5% CO₂ and 90% relative humidity in a carbon dioxide incubator. The experiment was carried out in triplicate.

After 24-hours of incubation, the working solutions of Compound 1 of different concentrations (100 μl) were added in the experimental wells and blanks whereas positive controls of required concentrations (100 μl) were added into wells for positive controls. The equivalent amount of complete growth medium was added to control set.

The plates were further incubated for 48-hours (at 37° C. in an atmosphere of 5% CO₂ and 90% relative humidity in a carbon dioxide incubator) after addition of test material etc. and then the cell growth was stopped by gently layering of 50 μl of TCA (50% trichloroacetic acid) on top of the medium in all the wells. The plates were incubated at 4° C. for one hour to fix the cells attached to the bottom of the wells. Liquids of all the wells were gently pipetted out and discarded. The plates were washed five times with distilled water to remove TCA, growth medium, low molecular weight metabolites, serum proteins etc. Plates were air dried.

Cell growth was measured by staining with sulforhodamine B dye (SRB). The SRB solution (100 μl of 0.4% SRB in 1% acetic acid) was added to each well and the plates were incubated at room temperature for 30 minutes. The unbound SRB was quickly removed by washing the wells five times with 1% acetic acid and plates were air dried. Tris-buffer (100 μl of 0.01M, pH 10.4) was added to all the wells and plates were gently stirred for 5 minutes on a mechanical stirrer. The optical density was recorded on ELISA reader at 540 nm.

The cell growth in presence of test material was determined by subtracting mean OD value of respective blank from the mean OD value of experimental set. Like wise, cell growth in absence of test material (control set) and in presence of positive control was also determined. The per cent cell growth in presence of test material was determined considering the cell growth in absence of test material as 100% and in turn per cent inhibition was calculated.

In vitro cytotoxicity of Compound 1 was determined against human cervix (SiHa), CNS (SK-N-MC), colon (HT-29), lung (A-549 & HOP-62), liver (Hep-2), ovary (OVCAR-5) and prostate (PC-3) representing different organs. The results are summarized in Table 5. Compound 1 showed dose dependent inhibition of cell growth of all the human cancer cell lines studied except HOP-62. The inhibition varied from 41-97% at 100 μg/ml. It was most effective against human prostate cancer cell line PC-3 and least effective against cervix cell line SiHa.

The structure of Compound 2 was elucidated as ent-pimmarane-7(8),9(11),15-triene-19 oic acid and is annexed herewith.

The GC-MS of Compound 2 showed the molecular ion peak at m/z 300 coresponding to molecular formula C₂₀H₂₈O₂. The compound responded to the Vanillin-H₂SO₄ test for terpenoids and bromocresol green test for carboxylic acids on TLC plates.

IR=νₘₐₓ 3520-2880 cm⁻¹ (OH), 1678 (CO), νₘₐₓ 1440, 1408, 1330, 1184, 1092, 910 and 756 cm⁻¹ (Tri. Subst. Double bond)

The ¹H-NMR spectrum (Table 3) of Compound 2 revealed the presence of an exocyclic di-substituted double bond by displaying an ABX pattern of coupled protons at δ 4.88 (1H, d, J=2.0 6 Hz, Ha-16), 4.91 (1H, d, J=5.0. Hz, Hb-16) and 4.94 (1H, t, J=5,2 Hz, Hx-15). The spectrum contained two singlets at δ 5.62 (1H, H-7) and 5.75 (1H, H-11) confirming the presence of two tri-substituted double bonds which are in confugation

Since the compound Compound 2 shows UV absorption at λₘₐₓ^{MeoH} 240 nm, it must possess a conjugated chromophore in the molecule.

Since the proton resonance signal of the tri substituted double bond appeared as a singlet the most plausible position for it is Δ 7,8 and Δ 9,11. The ¹³C-NMR (Table 4) resonance signals for the vinylic carbons appeared at δc 120.3 (C-7), δc 146.8 (C-8), δc-138.3 (C-9), δc 138.4 (C-11), 113.3 (C-16) and 147.5 (C-15). This was further substantiated by the chemical shift, multiplicity and coupling constants of allylic protons at C-6 and C-12 at δ2.31 (1H, dd, br, J=5,8, Hz, H-6) and 2.14 (1H, br, d, J=5 H-12).

The mass spectrum of the compound showed a facile loss of CO₂H and HCO₂H from the molecular ion resulting in the base peak fragment at m/z 257 and a strong peak at m/z 256.

The presence of carboxyl function was confirmed by preparation of methyl ester of LK-3 with diazomethane whose ¹H-NMR spectrum contained the resonance signal due to carboxyl methoxy protons at δ 3.63 (3H, s) and an downfield shift in the resoance signals of two methyls at δ 0.99 (3H, s) and 1.19 (3H, s).

In Vitro Cytotoxicity of Compound 2 Against Human Cancer Cell Lines:

The human cancer cell lines were grown, harvested and cytotoxicity was determined exactly as mentioned in Example 3 except that the test material used was Compound 2 that was dissolved in DMSO and three working test solutions were prepared of the same concentrations as in Example 3.

In vitro cytotoxicity of Compound 2 was determined against the same human cancer cell lines as in Example 3 representing different organs. The results are summarized in Table 5. Compound 2 showed dose dependent inhibition of cell growth of all the human cancer cell lines studied except HOP-62. The inhibition varied from 38-97% at 100 μg/ml. It was least effective against human cervix cancer cell line (SiHa) but was highly active against rest of the cell lines.

TABLE 1
¹H-NMR (chemical shifts) of Compound 1 (200 MHz, CD Cl₃ )

| δ | Integrated No. of Protons | Multiplicity | J in Hz | Assignment |
|---|---|---|---|---|
| 0.65 | 3 | S | — | H-18 |
| 1.01 | 3 | S | — | H-20 |
| 1.25 | 3 | S | — | H-17 |
| 2.16 | 2H | dd, br | 6, 5 | H-7 |
| 2.31 | 1H | dd, br | 6, 7 | H-9 |
| 4.90 | 1H | Dd | 6, 7 | Ha-16 |
| 4.95 | 1H | S | — | H-14 |
| 5.72 | 1H | Dd | 6, 7 | H-15 |

TABLE 2
¹³C-NMR of Compound 1, 500 MHz

| Carbon No. | δc | Carbon No. | δc |
|---|---|---|---|
| 1 | 39.3 | 11 | 19.6 |
| 2 | 19.3 | 12 | 36.5 |
| 3 | 37.98 | 13 | 38.5 |
| 4 | 44.1 | 14 | 128.0 |
| 5 | 56.2 | 15 | 147.2 |
| 6 | 24.2 | 16 | 113.0 |
| 7 | 35.8 | 17 | 29.4 |
| 8 | 138.0 | 18 | 29.2 |
| 9 | 50.6 | 19 | 184.5 |
| 10 | 38.56 | 20 | 13.3 |

TABLE 3
¹H-NMR chemical shifts of Compound 2 (200 MHz, CD Cl₃)

| | Integrated No. of | | | |
|---|---|---|---|---|
| δ | Protons | Multiplicity | J in Hz | Assignment |
| 0.65 | 3 | s | — | H-18 |
| 1.09 | 3 | s | — | H-20 |
| 110 | 3 | s | — | H-17 |
| 1.48-1.52 | 6H | m, br | — | H-1 |
| 1.38 | 1H | dd | 3.3, 7.3 | H-5 |
| 2.31 | 1H | dd, br | 5, 8 | Ha-6 |
| 5.62 | 1H | dd | 7, 4 | H-7 |
| 5.75 | 1H | dd | 7.3, 3.5 | H-11 |
| 2.14 | 1H | br, d | 5 | Ha-12 |
| 2.15 | 1H | d, br | 5 | Hb-12 |
| 2.04 | 2H | s, br | — | Ha-14 |
| 4.94 | 1H | t | 5, 2 | Hx-15 |
| 4.88 | 1H | d | 2.06 | Ha-16 |
| 4.91 | 1H | d | 5 | Hb-16 |

TABLE 4
¹³C-NMR of Compound 2, 500 MHz

| Carbon No. | δc | Carbon No. | δc |
|---|---|---|---|
| 1 | 39.6 | 11 | 138.4 |
| 2. | 19.6 | 12. | 38.9 |
| 3. | 36.2 | 13 | 38.3 |
| 4. | 44.4 | 14 | 30.1 |
| 5. | 56.5 | 15. | 147.5 |
| 6. | 32.3 | 16. | 113.3 |
| 7. | 120.3 | 17. | 29.5 |
| 8. | 146.18 | 18. | 29.7 |
| 9. | 138.2 | 19 | 185.2 |
| 10. | 39.6 | 20. | 14.1 |

TABLE 5
In vitro cytotoxicity of Compound 1 & 2 against human cancer cell lines

| | | Cell line & Tissue | | | | | | | |
|---|---|---|---|---|---|---|---|---|---|
| Test | | | | | SK-N- | | | | HOP- |
| ma- | Conc. | A-549 | Hep-2 | OVCAR-5 | MC | HT-29 | PC-3 | SiHa | 62 |
| terial | (μg/ml) | Lung | Liver | Ovary | CNS | Colon | Prostate | Cervix | Lung |
| | | (Growth inhibition, %) | | | | | | | |
| Com- | 10 | 13 | 20 | 00 | 00 | 02 | 05 | 00 | 00 |
| pound | 30 | 72 | 68 | 66 | 75 | 70 | 78 | 14 | 00 |
| 1 | 100 | 94 | 80 | 76 | 88 | 94 | 97 | 41 | 03 |
| Com- | 10 | 09 | 18 | 12 | 22 | 00 | 00 | 00 | 00 |
| pound | 30 | 71 | 70 | 67 | 85 | 70 | 83 | 8 | 00 |
| 2 | 100 | 97 | 91 | 96 | 92 | 95 | 94 | 38 | 00 |
| 5FU | 1 × | — | — | — | — | 43 | — | 17 | — |
| | 10⁻⁴M | | | | | | | | |
| Tamo- | 1 × | 19 | — | 00 | — | — | — | — | 02 |
| xifen | 10⁻⁵M | | | | | | | | |
| Tamo- | 1 × | 98 | — | 100 | — | — | — | — | 20 |
| xifen | 10⁻⁴M | | | | | | | | |
| Myto- | 1 × | | 87 | — | — | — | 78 | — | — |
| mycin C | 10⁻⁵M | | | | | | | | |
| 5 | | | | | | | | | |

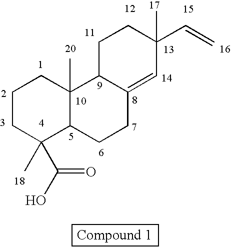

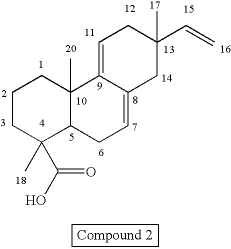

FLOW CHART

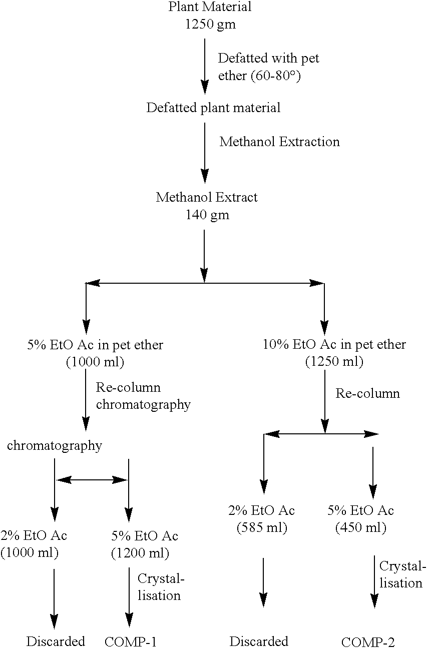

1. Novel compounds of formulae (1) and (2) named as ent-pimmarane 8(14),15-diene-19 oic acid and ent-pimmarane 7(8),9(11),15 triene-19 oic acid respectively, isolated from plant Lavatera cachmeriania.

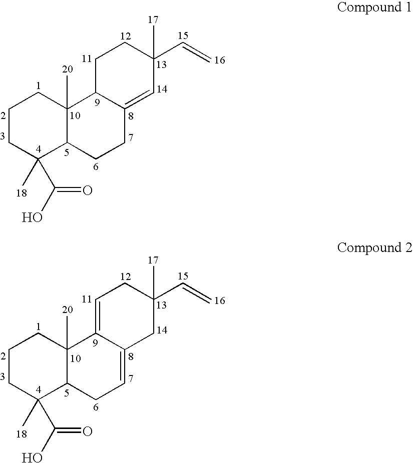

2. Compounds as claimed in claim 1 are having the following characteristics:
Compound (1):
m/z: 302(M+) IR₍νₘₐₓ₎: 3552-3228 (0-H); 1684 (C=0); 1598; 1410 and 910 (trisubstituted double bond) and 1264, 1024, 860 (di-substituted double bond as well) cm⁻¹ UV (λₘₐₓMeOH): 215 nm
¹H-NMR and ¹³C-NMR of compound (1):
TABLE 1
¹H-NMR (chemical shifts) of Compound 1 (200 MHz, CD Cl₃)

| | Integrated | | | |
|---|---|---|---|---|
| | No. of | | | |
| δ | Protons | Multiplicity | J in Hz | Assignment |
| 0.65 | 3 | S | — | H-18 |
| 1.01 | 3 | S | — | H-20 |
| 1.25 | 3 | S | — | H-17 |
| 2.16 | 2H | dd, br | 6, 5 | H-7 |
| 2.31 | 1H | dd, br | 6, 7 | H-9 |
| 4.90 | 1H | Dd | 6, 7 | Ha-16 |
| 4.95 | 1H | S | — | H-14 |
| 5.72 | 1H | Dd | 6, 7 | H-15 |

TABLE 2
¹³C-NMR of Compound 1, 500 MHz

| Carbon No. | δc | Carbon No. | δc |
|---|---|---|---|
| 1 | 39.3 | 11 | 19.6 |
| 2 | 19.3 | 12 | 36.5 |
| 3 | 37.98 | 13 | 38.5 |
| 4 | 44.1 | 14 | 128.0 |
| 5 | 56.2 | 15 | 147.2 |
| 6 | 24.2 | 16 | 113.0 |
| 7. | 35.8 | 17 | 29.4 |
| 8. | 138.0 | 18 | 29.2 |
| 9. | 50.6 | 19 | 184.5 |
| 10 | 38.56 | 20 | 13.3 |

Compound (2):
m/z: 300(M⁺)
IR₍νₘₐₓ₎ 3520-2880 (OH); 1678 (CO); νₘₐₓ 1440; 1408; 1330; 1184; 1092; 910 and 756 (tri-substituted double bond) cm⁻¹
UV (λₘₐₓ^{MeoH}): 240 nm
¹H-NMR and ¹³C-NMR of compound (2):
TABLE 3
¹H-NMR chemical shifts of Compound 2 (200 MHz, CD Cl₃)

| | Integrated No. | | | |
|---|---|---|---|---|
| δ | of Protons | Multiplicity | J in Hz | Assignment |
| 0.65 | 3 | s | — | H-18 |
| 1.09 | 3 | s | — | H-20 |
| 110 | 3 | s | — | H-17 |
| 1.48-1.52 | 6H | m, br | — | H-1 |
| 1.38 | 1H | dd | 3.3, 7.3 | H-5 |
| 2.31 | 1H | dd, br | 5, 8 | Ha-6 |
| 5.62 | 1H | dd | 7, 4 | H-7 |
| 5.75 | 1H | dd | 7.3, 3.5 | H-11 |
| 2.14 | 1H | br, d | 5 | Ha-12 |
| 2.15 | 1H | d, br | 5 | Hb-12 |
| 2.04 | 2H | s, br | — | Ha-14 |
| 4.94 | 1H | t | 5, 2 | Hx-15 |
| 4.88 | 1H | d | 2.06 | Ha-16 |
| 4.91 | 1H | d | 5 | Hb-16 |

TABLE 4
¹³C-NMR of Compound 2, 500 MHz

| Carbon No. | δc |
|---|---|
| 1 | 39.6 |
| 2. | 19.6 |
| 3. | 36.2 |
| 4. | 44.4 |
| 5. | 56.5 |
| 6. | 32.3 |
| 7. | 120.3 |
| 8. | 146.18 |
| 9. | 138.2 |
| 10. | 39.6 |
| 11 | 138.4 |
| 12. | 38.9 |
| 13 | 38.3 |
| 14 | 30.1 |
| 15. | 147.5 |
| 16. | 113.3 |
| 17. | 29.5 |
| 18. | 29.7 |
| 19 | 185.2 |
| 20. | 14.1 |

3. A process for the isolation of compounds of claim 1, the said process comprising steps of:
(a) powdering the plant part of Lavatera Cachmiriana,
(b) defatting the powdered plant of step(a), with an organic solvent,
(c) extracting the defatted plant-material of step (b) with an alcoholic solvent to obtain alcoholic extract,
(d) evaporating alcoholic extract of step (c), under reduced pressure to obtain a dry extract,
(e) purifying the dried extract of step (d) silica gel column chromatography, eluting with a mixture of organic solvents, and
(f) pooling identical eluted fractions of step (e) on the basis of TLC analysis, concentrating the pooled fraction to reduced volume and crystallizing by adding an organic solvent to obtain the required compounds (1) and (2).

4. A process of claim 1, wherein in step (a) the plant part used, is whole plant.

5. A process of claim 1, wherein in step (b) the organic solvent used, is selected from petroleum ether or hexane.

6. A process of as claimed in claim 5, wherein the solvent used is petroleum ether.

7. A process of claim 1, wherein in step (b) the w/v ratio of plant material and organic solvent ranges from 1:20 to 25.

8. A process of claim 1, wherein in step (c) the alcoholic solvent used is C₁ to C₆ aliphatic alcohol selected from a group consisting of methanol, ethanol, isopropanol, butanol, pentanol and hexanol.

9. A process of claim 8, wherein in the alcoholic solvent used is methanol.

10. A process of claim 1, wherein in step (c) the w/v ratio of plant material and alcoholic solvent ranges from 1:15 to 20.

11. A process of claim 1, wherein in step (e) the mixture of organic solvent used for elution is a mixture of petroleum ether: ethylacetate.

12. A process of claim 1, wherein the v/v percentage of petroleum ether:ethylacetate ranges from 98:2 to 90:10

13. A process of claim 12, wherein petroleum ether:ethylacetate (95:15) eluate provide compound (1).

14. A process of claim 12, wherein petroleum ether:ethylacetate (90:10) eluate provides compound (2).

15. A process of claim 1, wherein in step (f) the organic solvent used for crystallization, is petroleum ether.

16. Pharmaceutical composition comprising an effective amount compound (1) named as ent-pimmarane 8(14),15-diene-19 oic acid or compound (2) named as ent-pimmarane 7(8),9(11),15 triene-19 oic acid or combination thereof, for treating a said subject afflicted with cancer selected from group consisting of lungs, ovary, central nervous system (CNS), colon, prostrate and cervix.

17. The composition as claimed in claim 16, wherein the compounds (1) and/or (2) may be administered singly or in combination with pharmaceutically acceptable excipient.

18. The composition as claimed in claim 17, wherein the pharmaceutically acceptable excipient used may be an additive, carrier, diluent, solvent, filler, lubricant, excipient, binder or stabilizer.

19. The composition as claimed in claim 16, wherein compounds (1) and/or (2) may be administered orally or systemically.

20. The composition as claimed in claim 16, wherein the pathological conditions treated arising from cancer afflicted lungs, ovary, central nervous system (CNS), colon, prostrate, and cervix.

21. The composition as claimed in claim 16, wherein the said subjects are selected from animals, mammals or humans; preferably humans.

22. The composition as claimed in claim 16, wherein the composition containing compound (1) inhibits the growth of human lung cancer cells (A-549) in the range of 13 to 94% at a concentration range of 10 to 100 μg/ml.

23. The composition as claimed in claim 16, wherein the composition containing compound (1) inhibits the growth of human liver cancer cells (Hep-2) in the range of 18 to 91% at a concentration range of 10 to 100 μg/ml.

24. The composition as claimed in claim 16, wherein the composition containing compound (1) inhibits the growth of human ovary cancer cells (OVCAR-5) in the range of 0 to 76% at a concentration range of 10 to 100 μg/ml.

25. The composition as claimed in claim 16, wherein the composition containing compound (1) inhibits the growth of human CNS cancer cells (SK-N-MC) in the range of 0 to 88% at a concentration range of 10 to 100 μg/ml.

26. The composition as claimed in claim 16, wherein the composition containing compound (1) inhibits the growth of human colon cancer cells (HT-29) in the range of 2 to 94% at a concentration range of 10 to 100 μg/ml.

27. The composition as claimed in claim 16, wherein the composition containing compound (1) inhibits the growth of human prostrate cancer cells (PC-3) in the range of 5 to 97% at a concentration range of 10 to 100 μg/ml.

28. The composition as claimed in claim 16, wherein the composition containing compound (1) inhibits the growth of human cervix cancer cells (Si Ha) in the range of 0 to 41% at a concentration range of 10 to 100 μg/ml.

29. The composition as claimed in claim 16, wherein the compound (2) inhibits the growth of human lung cancer cells (A-549) in the range of 9 to 97% at a concentration range of 10 to 100 μg/ml.

30. The composition as claimed in claim 16, wherein the composition containing compound (2) inhibits the growth of human liver cancer cells (hep-2) in the range of 18 to 91% at a concentration range of 10 to 100 μg/ml.

31. The composition as claimed in claim 16, wherein the composition containing compound (2) inhibits the growth of human ovary cancer cells (OVCAR-5) in the range of 12 to 96% at a concentration range of 10 to 100 μg/ml.

32. The composition as claimed in claim 16, wherein the composition containing compound (2) inhibits the growth of human CNS cancer cells (SK-N-MC) in the range of 22 to 92% at a concentration range of 10 to 100 μg/ml.

33. The composition as claimed in claim 16, wherein the composition containing compound (2) inhibits the growth of human colon cancer cells (HT-29) in the range of 0 to 95% at a concentration range of 10 to 100 μg/ml.

34. Use of compounds (1) and (2) as claimed in claim 1, for treating a said subject afflicted with cancer mainly of lungs, ovary, central nervous system (CNS), colon, prostrate, and cervix; said method comprising administering a pharmaceutically effective dosage of compound (1) and/or (2) or a composition containing compound (1) or (2) or a combination thereof to the said subject.

35. The use of compounds as claimed in claim 34, wherein the compounds (1) and/or (2) may be administered singly or in combination with pharmaceutically acceptable excipient.

36. The use of compounds as claimed in claim 35, wherein the pharmaceutically acceptable excipient used may be an additive, carrier, diluent, solvent, filler, lubricant, excipient, binder or stabilizer.

37. The use of compounds as claimed in claim 34, wherein compounds (1) and/or (2) may be administered orally or systemically.

38. The use of compounds as claimed in claim 35, wherein the pathological conditions treated arising from cancer afflicted lungs, ovary, central nervous system (CNS), colon, prostrate, and cervix.

39. The use of compounds as claimed in claim 34, wherein the said subjects are selected from animals, mammals or humans; preferably humans.

40. The use of compounds as claimed in claim 34, wherein the compound (1) inhibits the growth of human lung cancer cells (A-549) in the range of 13 to 94% at a concentration range of 10 to 100 μg/ml.

41. The use of compounds as claimed in claim 34, wherein the compound (1) inhibits the growth of human liver cancer cells (Hep-2) in the range of 18 to 91% at a concentration range of 10 to 100 μg/ml.

42. The use of compounds as claimed in claim 34, wherein the compound (1) inhibits the growth of human ovary cancer cells (OVCAR-5) in the range of 0 to 76% at a concentration range of 10 to 100 μg/ml.

43. The use of compounds as claimed in claim 34, wherein the compound (1) inhibits the growth of human CNS cancer cells (SK-N-MC) in the range of 0 to 88% at a concentration range of 10 to 100 μg/ml.

44. The use of compounds as claimed in claim 34, wherein the compound (1) inhibits the growth of human colon cancer cells (HT-29) in the range of 2 to 94% at a concentration range of 10 to 100 μg/ml.

45. The use of compounds as claimed in claim 34, wherein the compound (1) inhibits the growth of human prostrate cancer cells (PC-3) in the range of 5 to 97% at a concentration range of 10 to 100 μg/ml.

46. The use of compounds used as claimed in claim 34, wherein the compound (1) inhibits the growth of human cervix cancer cells (Si Ha) in the range of 0 to 41% at a concentration range of 10 to 100 μg/ml.

47. The use of compounds as claimed in claim 34, wherein the compound (2) inhibits the growth of human lung cancer cells (A-549) in the range of 9 to 97% at a concentration range of 10 to 100 μg/ml.

48. The use of compounds as claimed in claim 34, wherein the compound (2) inhibits the growth of human liver cancer cells (hep-2) in the range of 18 to 91% at a concentration range of 10 to 100 μg/ml.

49. The use of compounds as claimed in claim 34, wherein the compound (2) inhibits the growth of human ovary cancer cells (OVCAR-5) in the range of 12 to 96% at a concentration range of 10 to 100 μg/ml.

50. The use of compounds as claimed in claim 34, wherein the compound (2) inhibits the growth of human CNS cancer cells (SK-N-MC) in the range of 22 to 92% at a concentration range of 10 to 100 μg/ml.

51. The use of compounds as claimed in claim 34, wherein the compound (2) inhibits the growth of human colon cancer cells (HT-29) in the range of 0 to 95% at a concentration range of 10 to 100 μg/ml.
